(19) United States
(12) Patent Application Publication
(10) Pub. No.: US 2021/0398614 A1
(43) Pub. Date: Dec. 23, 2021

(54) METHODS AND SYSTEMS FOR CREATING GROUPS OF AQUATIC ORGANISMS BASED ON PREDICTED GROWTH POTENTIAL

(71) Applicant: ARIZONA BOARD OF REGENTS ON BEHALF OF THE UNIVERSITY OF ARIZONA, Tucson, AZ (US)

(72) Inventor: Benjamin J. Renquist, Tucson, AZ (US)

(21) Appl. No.: 17/395,203

(22) Filed: Aug. 5, 2021

Related U.S. Application Data
(63) Continuation-in-part of application No. 15/754,126, filed on Feb. 21, 2018, now abandoned.
(60) Provisional application No. 62/208,433, filed on Aug. 21, 2015.

(51) Int. Cl. G16B 20/40; G16B 45/00

(52) U.S. Cl. CPC G16B 20/40; G01N 33/52; G16B 45/00

(57) ABSTRACT

Methods and systems, including computer implemented methods and systems, for creating groups of embryonic fish or juvenile aquatic organisms from a pool based on predicted growth potential or feed efficiency wherein whole-body metabolic rate of each individual in the pool is measured and individuals are sorted into one of two or more containers based on relative whole-body metabolic rate. The embryonic fish or juvenile aquatic organisms with the highest whole-body metabolic rate are predicted to grow significantly more than those with the lowest whole-body metabolic rate.

### CROSS REFERENCE

This application is a continuation-in-part and claims benefit of U.S. patent application Ser. No. 15/754,126, filed Feb. 21, 2018, which is a 371 application and claims benefit of PCT/US16/48006, filed Aug. 22, 2016, which claims benefit of U.S. Provisional Patent Application No. 62/208,433, filed Aug. 21, 2015, the specification(s) of which is/are incorporated herein in their entirety by reference.

### GOVERNMENT SUPPORT

This invention was made with government support under Grant No. 2010-38500-21758 awarded by USDA/NIFA. The government has certain rights in the invention.

### FIELD OF THE INVENTION

The present invention relates to methods for organizing, creating, preparing, or separating groups of aquatic organisms from a pool, e.g., embryonic fish, juvenile aquatic organisms, etc., based on relative predicted growth potential or feed efficiency, e.g., relative to the pool of organisms.

### BACKGROUND OF THE INVENTION

In fish species, growth is positively correlated with energy expenditure. Current methods of measuring metabolic rate in fish species involve measuring oxygen consumption, e.g., measuring oxygen consumption per unit of body weight. However, measuring oxygen consumption requires specialized equipment, training so as to limit fish stress, and an understanding of factors that influence metabolic rate (e.g., feeding status, hormonal status).

A review of maternal effects in fish populations (Green, 2008, Advances in Marine Biology 54:1-105) discusses that female identity explained a large proportion of variation in egg diameter and in hatching length (e.g., the size of the mother could largely predict the size of the hatchling). Heath and colleagues (Heath et al., 1999, Evolution 53(5):1605-1611) studied the maternal effect on progeny growth and found that by 180 days, there was no detectable difference between the progeny that were initially bigger due to maternal effect and the progeny that were not. Thus, using size and weights of females, embryos, or hatchlings, one cannot necessarily predict how large fish will be beyond the maternal effect time period (e.g., 2 months). It was surprisingly discovered that measuring metabolic rate allowed for the prediction of growth potential beyond the time frame associated with the maternal effect (e.g., to harvest size), and that high metabolic rates were indicative of high growth potential. Further, in warm-blooded animals, high metabolic rates are associated with slower growth. It was surprisingly discovered that in fish, high metabolic rates are associated with high growth. In oysters, inbred, slow growing lines have a higher metabolic rate than outcrossed fast growing families.

The present invention features methods and systems (e.g., a colorimetric/fluorescent methods and systems) for selecting, preparing, creating, or generating groups of embryonic fish or juvenile aquatic organisms from a pool based on predicted relative growth potential or feed efficiency.

The methods and systems of the present invention may be used to assess the genetic potential for growth of the plant or animal. For example, the methods and systems may be used to predict the growth potential of a plant or animal. Measuring the metabolic rate of said plant or animal may be helpful for identifying and selecting individuals within a group that have greater predicted growth potential, e.g., individuals that are most likely to grow faster and/or larger. The methods and systems of the present invention may also be used to segregate fast and slow growing fish. These applications may be beneficial for the aquaculture industry, e.g., hatcheries, fish farms or the like. For example, without wishing to limit the present invention to any theory or mechanism, it is believed that the methods and systems of the present invention, which may allow for selection of genetically superior brood stock, may have a positive impact on profitability given that selecting for genetic potential for growth currently has been limited by (a) interactions between aggression and growth, (b) inability to select in wild-caught brood stock, and (c) the long generation interval in slow maturing species.

Without wishing to limit the present invention to any theory or mechanism, it is believed that the methods and systems of the present invention may help eliminate the need for special equipment for measuring oxygen consumption and minimize the effects of external factors (feeding/hormonal status).

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

The present invention features an automated computer implemented method for preparing groups of embryonic fish or juvenile aquatic organisms from a pool based on predicted growth potential or feed efficiency.

In some embodiments, the method comprises measuring whole-body metabolic rate of an individual in the pool of embryonic fish or juvenile aquatic organisms, wherein metabolic rate is measured by detection of an indicator, e.g., a colorimetric/fluorescent redox indicator); and moving the individual to a first container if the whole-body metabolic rate is above a precalculated threshold of fluorescence or moving the individual to a container different from the first container (e.g., a second container, a third container, one of several other containers, etc.) if the whole-body metabolic rate is below a precalculated threshold of fluorescence. The method may comprise repeating the above steps for all or a portion of the individuals in the pool. The precalculated threshold may be determined by measuring whole-body metabolic rate in a portion of the other individuals in the pool. The precalculated threshold may change (e.g., the threshold may be recalculated) after the reading of each individual.

The embryonic fish or juvenile aquatic organisms may be aquaculturally farmed organisms. In some embodiments, the embryonic fish are salmon, trout, zebrafish, wea bass, tilapia, catfish, sole, or cobia. The present invention is not limited to the aforementioned examples of embryonic fish. In some embodiments, the juvenile aquatic organisms are shrimp, lobster, prawn, crab, or tiger prawn. The present invention is not limited to the aforementioned aquatic organisms.

The present invention also provides an automated computer implemented system for preparing groups of embryonic fish or juvenile aquatic organisms from a pool based on predicted growth potential or feed efficiency.

In some embodiments, the system comprises a detector for detecting an indicator (e.g., a colorimetric/fluorescent redox indicator) in an individual of the pool. The indicator may be for measuring whole-body metabolic rate of the individual, e.g., in the form of a “detection value” or “fluorescence value” for the individual (note that the term “fluorescence value” may simply refer to the measurement by the detector and may not necessarily be limited to fluorescence since not all indicators may be fluorescent indicators).

The system may further comprise a sorting component (e.g., robotic arm, tubing, hydraulic mechanisms, valves, etc.) for moving an individual of the pool to one of two or more containers after detection of the redox indicator. The system may comprise two containers. In some embodiments, the system comprises three containers. In some embodiments, the system comprises four containers. In some embodiments, the system comprises five containers. In some embodiments, the system comprises more than five containers. In some embodiments, the system comprises from 2 to 10 containers.

The system may further comprise a microprocessor operatively connected to the detector and the sorting component. The microprocessor receives input signals from the detector as fluorescence values (or “detection values”) of individuals of the pool. The microprocessor sends output signals to the sorting component to move the individuals to one of the two or more containers. In some embodiments, the microprocessor is configured to calculate a threshold (e.g., a “precalculated threshold”) of fluorescence (or whichever indicator is used) based on input from the detector; the precalculated threshold of fluorescence refers to a minimum fluorescence value (or detection value) that would be indicative of a predicted high metabolic rate individual. In some embodiments, the microprocessor is configured to send an output signal to the sorting component to move the individual to a first container if the individual has a fluorescence value higher than the precalculated threshold of fluorescence or send an output signal to the sorting component to move the individual to a container different from the first container (e.g., a second container, a third container, a fourth container, etc.) if the individual has a fluorescence value below the precalculated threshold of fluorescence. The particular container chosen may be based on the fluorescence value (or detection value) of the individual relative to the precalculated threshold and/or other thresholds.

In some embodiments, the system allows for a user to help determine the threshold values or how to separate the individuals relative to each other's predicted metabolic rate.

As previously discussed, the embryonic fish or juvenile aquatic organisms may be aquaculturally farmed organisms. In some embodiments, the embryonic fish are salmon, trout, zebrafish, wea bass, tilapia, catfish, sole, or cobia. The present invention is not limited to the aforementioned examples of embryonic fish. In some embodiments, the juvenile aquatic organisms are shrimp, lobster, prawn, crab, or tiger prawn. The present invention is not limited to the aforementioned aquatic organisms.

The present invention also provides a computer implemented method for preparing groups of embryonic fish or juvenile aquatic organisms from a pool based on predicted growth potential or feed efficiency.

In some embodiments, the method comprises introducing to the pool of embryonic fish or juvenile aquatic organisms an indicator (e.g., a colorimetric/fluorescent redox indicator) of whole-body metabolic rate (e.g., fluorescence or emission of the redox indicator is for determining relative whole-body metabolic rate).

In some embodiments, the method comprises introducing the pool of embryonic fish or juvenile aquatic organisms to an automated system (e.g., as described herein), wherein the automated system sequentially detects fluorescence values (or “detection values”) of n embryonic fish or juvenile aquatic organisms (E₁-Eₙ) and calculates a threshold fluorescence value V corresponding to a minimum fluorescence value (or minimum detection value) within a X % highest fluorescence values detected. In some embodiments, n and X are pre-programmed by a user.

The method may further comprise sequentially detecting a fluorescence value (or “detection value”) of an additional individual, e.g., an additional embryonic fish or juvenile aquatic organism (Eₙ₊₁) and moving Eₙ₊₁ to a first container if the fluorescence value is greater than V or a container different from the first container if the fluorescence value is less than V.

The method may further comprise recalculating a new V based on fluorescence values of the previous n embryonic fish or juvenile aquatic organisms or a portion thereof.

The method may further comprise detecting a fluorescence value of a new embryonic fish or juvenile aquatic organism and moving the new embryonic fish or juvenile aquatic organism to a first container if the fluorescence value is greater than the new V or container different from the first container if the fluorescence value is less than the new V.

In some embodiments, the method repeats the steps: recalculating a new V based on fluorescence values of the previous n embryonic fish or juvenile aquatic organisms (or a portion thereof); and detecting a fluorescence value of a new embryonic fish or juvenile aquatic organism and moving the new embryonic fish or juvenile aquatic organism to a first container if the fluorescence value is greater than the new V or a second container if the fluorescence value is less than the new V.

As previously discussed, n and X may be pre-programmed by a user. As an example, a user can decide if he/she wants individuals that fall into the top 25% of relative whole-body metabolic rate to be moved to a first container and the remaining individuals to go to a second container. As another example, a user can decide if he/she wants individuals that fall into the top 10% of relative whole-body metabolic rate to be moved to a first container, the individuals that fall into the 10-50% to go to a second container, and the remaining individuals to go to a third container. The user may choose to apply the method to 1,000 individuals, 5,000 individuals, 10,000 individuals, etc. In some embodiments, n is 500. In some embodiments, n is from 10 to 1,000. In some embodiments, n is from 1,000 to 10,000. In some embodiments, n is from 10,000 to 100,000.

In some embodiments, the detector is a visual detector, a photodetector, a photodiode detector, a fluorescent detector, or a camera. In some embodiments, the system comprises two or more containers. In some embodiments, the system comprises two containers, three containers, four containers, five containers, six containers, seven containers, eight containers, nine containers, or ten containers.

As previously discussed, embryonic fish or juvenile aquatic organisms may be aquaculturally farmed organisms. In some embodiments, the embryonic fish are salmon, trout, zebrafish, wea bass, tilapia, catfish, sole, or cobia. The present invention is not limited to the aforementioned examples of embryonic fish. In some embodiments, the juvenile aquatic organisms are shrimp, lobster, prawn, crab, or tiger prawn. The present invention is not limited to the aforementioned aquatic organisms.

The present invention also provides a computer implemented sorting system for preparing groups of embryonic fish or juvenile aquatic organisms from a pool into groups based on predicted growth potential or feed efficiency.

In some embodiments, the system comprises a detector for detecting an indicator, e.g., a colorimetric/fluorescent redox indicator, in an individual of the pool so as to measure whole-body metabolic rate (e.g., to measure a fluorescence value or a detection value) for the individual. In some embodiments, the system comprises a sorting component for moving an individual of the pool to a container, e.g., a first container, a second container, one container out of c containers (c referring to the number of containers), etc., after detection of the redox indicator. Note that there are at least two containers (e.g., c is at least two).

The system further comprises a microprocessor operatively connected to the detector and the sorting component. The microprocessor receives input signals from the detector as fluorescence values of individuals of the pool and sends output signals to the sorting component to move the individuals to one of c containers.

In some embodiments, the microprocessor is configured to receive and store fluorescence values (or detection values) from the detector as the detector sequentially detects light emitted from n embryonic fish or juvenile aquatic organisms (E₁ through Eₙ), wherein n is pre programmed by a user, wherein n is at least 10.

In some embodiments, the microprocessor is configured to organize a distribution of fluorescence values (or detection values) of all or a portion of the n detected embryonic fish or juvenile aquatic organisms.

In some embodiments, the microprocessor is configured to calculate V, wherein V refers to the minimum fluorescence value corresponding to the X % highest fluorescence values (or detection values) in the distribution, wherein X is pre-programmed by a user.

In some embodiments, the microprocessor is configured to receive and store a fluorescence value (or detection value) from the detector when the detector detects light emitted from a new embryonic fish or juvenile aquatic organism (Eₙ₊₁) and (i) send a first output signal to the sorting component to direct Eₙ₊₁ to a first container if the fluorescence value (or detection value) of Eₙ₊₁ is above V, or (ii) send a second output signal to the sorting mechanism to direct Eₙ₊₁ to a container different from the first container if the fluorescence value (or detection value) of Eₙ₊₁ is below V.

In some embodiments, the microprocessor is configured to recalculate a new V based on fluorescence values (or detection values) of the previous n individuals (e.g., embryonic fish, juvenile aquatic organisms, mollusks, etc.), or a portion thereof.

In some embodiments, the microprocessor is configured to recalculate a new V based on fluorescence values of the previous n embryonic fish or juvenile aquatic organisms, or a portion thereof.

In some embodiments, the number of containers (e.g., “c”) is at least 3. In some embodiments, the number of containers (e.g., “c”) is from 2 to 10.

In some embodiments, n=500. In some embodiments, n is from 10 to 10,000. In some embodiments, n is 500. In some embodiments, n is from 10 to 1,000. In some embodiments, n is from 1,000 to 10,000. In some embodiments, n is from 10,000 to 100,000.

In some embodiments, the detector is a visual detector, a photodetector, a photodiode detector, a fluorescent detector, or a camera.

As previously discussed, the embryonic fish may be aquaculturally farmed organisms. The juvenile aquatic organisms may be aquaculturally farmed organisms. In some embodiments, the embryonic fish are salmon, trout, zebrafish, wea bass, tilapia, catfish, sole, or cobia. The present invention is not limited to the aforementioned examples of embryonic fish. In some embodiments, the juvenile aquatic organisms are shrimp, lobster, prawn, crab, or tiger prawn. The present invention is not limited to the aforementioned aquatic organisms.

### BRIEF DESCRIPTION OF THE DRAWINGS

The features and advantages of the present invention will become apparent from a consideration of the following detailed description presented in connection with the accompanying drawings in which:

FIG. 1 shows growth rates of embryos segregated into high and low metabolic rate quartiles. *P<0.05.

FIG. 2 shows body length vs. metabolic rate. Body length is similar in high and low metabolic rate embryonic fish on the day the assay is performed. This establishes that this is not merely selecting larger embryos.

FIG. 3A-3D show that changes in fluorescence are highly correlated with caudal fin (FIG. 3A) and skeletal muscle (FIG. 3B) explant mass. This establishes that within an animal, one can correct fluorescence change by tissue mass. Changes in fluorescence increase with duration of incubation with caudal fin (FIG. 3C) and skeletal muscle (FIG. 3D) explants. This indicates that the tissue remains viable for up to 6 h after collection.

FIG. 4A-4B relate to isolated cells from skeletal muscle and fin cell explants. FIG. 4A: Skeletal muscle satellite cells differentiate into proliferating myoblasts. FIG. 4B: Using caudal fin derived cells, the percent confluence was varied to create a range of total metabolic rates within a well. Fluorescent change from baseline, increased with # of fin cells in a well (P<0.0001).

FIG. 5 shows an analysis of RGB spectrum from wells (G3-G10) in an experiment using AlamarBlue® reduction methods (pink, purple and blue wells were indicative of embryos with high, moderate, and low metabolic rates, respectively.) Care was taken to ensure measurement was taken neither in a shadow nor ray of light. This preliminary analysis suggests that digital photograph analysis of red spectrum intensity can be used to quantitatively sort wells based on color change. Colors of dots for each well represent the exact RGB spectrum from the analysis.

FIG. 6 shows a histogram displaying the frequency distribution of metabolic rates measured by change in fluorescence. Note the long right tail composed of fish that have a high selective differential from the mean.

FIG. 7 shows comparisons between high and low metabolic rate fish made at identical weights. Slope within the metabolic rate group will determine the relationship between current body weight and explant metabolic rate. Body weight data is from fish that are already selected; thus, selection of fish with similar masses in each group may be feasible.

FIG. 8A-8C show the growth advantage in tilapia that had a high metabolic rate (top quartile) as embryos relative to those embryos that have a low metabolic rate (bottom quartile). The percent increase in growth attributable to the high metabolic rate is shown at each time point for each group (FIG. 8A is group 1, FIG. 8B is group 2, and FIG. 8C is group 3). At all time points significant differences existed.

FIG. 9A-9C show AlamarBlue reduction results in lettuce seeds. Lettuce seeds were sprouted in either fertilized or distilled water then transferred to either fertilized or distilled water 3 days later for analysis of AlamarBlue reduction. Plants placed into fertilized water showed increased AlmarBlue reduction at both 3 hours (FIG. 9A) and 24 hours (FIG. 9B) relative to plants that were placed into distilled water. Of note those plants that were sprouted in distilled water did generate significantly less signal in fertilized water during the first 3 hours of incubation than lettuce sprouts that were always in fertilized water. Also shown in FIG. 9C is the signal increase with duration of exposure of the lettuce sprout to AlamarBlue, which is an indication of the cumulative nature of this assay. This figure suggests that this assay can be used to test the nutrient quality of water or soil.

FIG. 10A-10B show representative results from Oyster Spat. FIG. 10A shows that oyster spat derived from different families induced nearly 3-fold differences in AlamarBlue reduction. This suggests that significant variation in NADH₂ production exists between families. FIG. 10B shows that if oyster spat within a cross are size separated, those spat that grow more slowly (runts; R) induce less signal than oysters that grow more quickly. This may likely be due to their lower tissue mass.

FIG. 11A-11D show that in both sized (FIG. 11A, FIG. 11B) and runt oysters (FIG. 11C, FIG. 11D) the signal generated increases with time and with the number of oysters within a well. Larger oysters (sized) generate signal more quickly and with fewer oysters than do runt oysters.

FIG. 12 shows D-larvae (2-day post fertilization larval oysters) increase the signal generated in this AlamarBlue based assay linearly as the number of larvae within a well increases (R²=0.9752).

FIG. 13A-13D show changes in fluorescence with various numbers of tilapia (FIG. 13A), trout (FIG. 13B), oysters (FIG. 13C), and shrimp (FIG. 13D) in a set volume. FIG. 13A-13D show that metabolic rate within a well (given volume) increases as the number of individuals within that well (volume) increases.

FIG. 14 shows that selection of tilapia broodstock based on metabolic rate, as measured by the AlamarBlue based assay, results in offspring with higher feed efficiency than offspring from Tilapia broodstock selected based on growth.

FIG. 15 shows the variability in metabolic rate within a family. This proposes that broodstock selection based on family selection will not improve genetics for growth nearly as rapidly as selection based on the individual metabolic rate.

FIG. 16 shows that slow growing inbred oyster lines (adam×adam) and (eve×eve) have higher metabolic rates than their outcrossed faster growing half-siblings (adam×eve).

FIG. 17 shows experimental evidence that high metabolic rate rainbow trout eggs (within a clutch) become fish that grow more quickly than low metabolic rate rainbow trout eggs. These sorts occurred within a clutch resulting in less difference than would occur if selection was from embryos across multiple clutches.

FIG. 18 shows that high metabolic rate Nile tilapia embryos grow more quickly than low metabolic rate Nile tilapia embryos. These sorts occurred within a clutch resulting in less difference than would occur if selection was from embryos across multiple clutches

FIG. 19 shows that high metabolic rate red tilapia embryos (within a clutch) grow more quickly than low metabolic rate red tilapia embryos. These sorts occurred within a clutch resulting in less difference than would occur if selection was from embryos across multiple clutches.

FIG. 20 establishes that the assay can also be applied to confirm known growth traits of fish. Herein, there are eggs from broodstock that were selected to grow quickly (A and C) and eggs from broodstock that were selected to have a strong immune system (B and D). Importantly, eggs in family A and B were the same temperature units, while eggs in families C and D were the same temperature units. This shows that eggs from growth-selected fish have a higher metabolic rate than eggs from immune system selected fish. It is also seen that eggs with more temperature days create more signal than those with lower temperature days (younger).

### DETAILED DESCRIPTION OF THE INVENTION

The present invention features methods and systems (e.g., a colorimetric/fluorescent methods and systems) for generating (e.g., preparing) groups of embryonic fish or juvenile aquatic organisms based on predicted growth potential or feed efficiency.

The present invention is not limited to the examples described herein.

Embodiments of the present invention can be freely combined with each other if they are not mutually exclusive.

### EXAMPLE 1

The present invention features methods and systems (e.g., a colorimetric/fluorescent methods and systems) for measuring metabolic rate in plants or aquatic animal species (e.g., embryonic fish, zebrafish, tilapia, trout, etc.). For example, the present invention features measuring over a period of time (e.g., 1 to 72 hours) the production of NADH₂, which is a product of metabolism and thus a direct indicator of the flux of metabolites through metabolite oxidation. The methods and systems of the present invention may monitor NADH₂ using a redox indicator (e.g., resazurin/resorufin or other appropriate indicator (e.g., NADH₂ indicator) such as but not limited to tetrazolium dyes, e.g., MTT, XTT, MTS, WST). The redox indicator is reduced by NADH₂, resulting in a colorimetric and fluorescent shift in solution. In some embodiments, the redox indicator remains reduced, and therefore can be used to provide a cumulative measurement of energy expenditure over a time period.

A review of maternal effects in fish populations (Green, 2008, Advances in Marine Biology 54:1-105) discusses that female identity explained a large proportion of variation in egg diameter and in hatching length (e.g., the size of the mother could largely predict the size of the hatchling). Heath and colleagues (Heath et al., 1999, Evolution 53(5):1605-1611) studied the maternal effect on progeny growth and found that by 180 days, there was no detectable difference between the progeny that were initially bigger due to maternal effect and the progeny that were not. Thus, using size and weights of females, embryos, or hatchlings, one cannot necessarily predict how large fish will be beyond the maternal effect time period (e.g., 2 months). It was surprisingly discovered that measuring metabolic rate allowed for the prediction of growth potential beyond the time frame associated with the maternal effect (e.g., to harvest size), and that high metabolic rates were indicative of high growth potential. Further, in warm-blooded animals, high metabolic rates are associated with slower growth. It was surprisingly discovered that in fish, high metabolic rates are associated with high growth. In oysters, inbred, slow growing lines have a higher metabolic rate than outcrossed fast growing families.

The methods and systems of the present invention may be used to assess the genetic potential for growth of the plant or animal. For example, the methods and systems may be used to predict the growth potential of a plant or animal. Measuring the metabolic rate of said plant or animal may be helpful for identifying and selecting individuals within a group that have greater predicted growth potential, e.g., individuals that are most likely to grow faster and/or larger. For example, fish with a high metabolic rate as embryos may weigh more than 30% more at eight months as compared to fish that have low metabolic rates as embryos. The methods and systems of the present invention may also be used to segregate fast and slow growing fish. These applications may be beneficial for the aquaculture industry, e.g., hatcheries, fish farms or the like. For example, without wishing to limit the present invention to any theory or mechanism, it is believed that the methods and systems of the present invention, which may allow for selection of genetically superior brood stock, may have a positive impact on profitability given that selecting for genetic potential for growth currently has been limited by (a) interactions between aggression and growth, (b) inability to select in wild-caught brood stock, and (c) the long generation interval in slow maturing species.

Without wishing to limit the present invention to any theory or mechanism, it is believed that the methods and systems of the present invention may help eliminate the need for special equipment (e.g., for measuring oxygen consumption), decrease variability of measures, and minimize the effects of external factors (feeding/hormonal status).

The present invention is not limited to use in aquatic animal species (e.g., embryonic fish, zebrafish, tilapia, trout, etc., with the ability to work with tissue explants and/or primary cells).

The present invention may also be used in plants. For example, the present invention may be used to test soil, water, and/or fertilizers. In some embodiments, the plants with the best genetics for growth may be selected. In some embodiments, water quality or soil quality is assessed. In some embodiments, the ability of different fertilizers to enhance growth is assessed.

The present invention features methods and systems for measuring growth rate in plant or aquatic animal species. The methods and systems of the present invention may be used to predict growth potential of a plant or animal. Measuring the growth rate of said plant or animal may be helpful for identifying and selecting individuals within a group that have greater growth potential, e.g., individuals that are most likely to grow faster and/or larger.

The methods for measuring growth rate (and/or for predicting growth potential) comprise measuring (over a period of time) the production of NADH₂ using a redox indicator (e.g., resazurin/resorufin or other appropriate indicator). The redox indicator is reduced by NADH₂, resulting in a colorimetric and fluorescent shift in solution (e.g., using AlamarBlue® reduction, pink, purple and blue wells may be indicative of embryos with high, moderate, and low metabolic rates, respectively). The redox indicator remains reduced, and therefore can be used to provide a cumulative measurement of energy expenditure over a time period. Redox indicators may include but are not limited to resazurin, a tetrazolium dye (e.g., MTT, XTT, MTS, WST), PrestoBlue®, or any other appropriate NADH₂ production indicator.

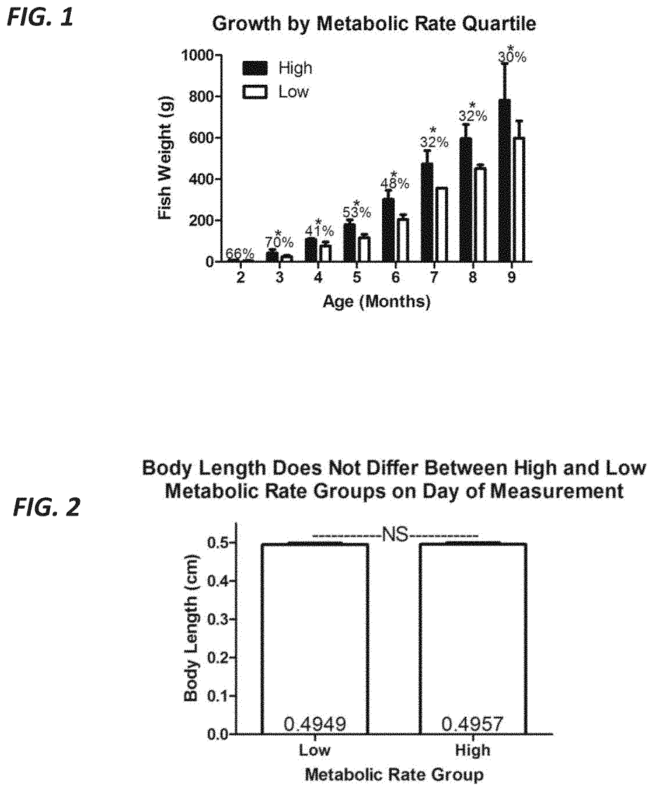

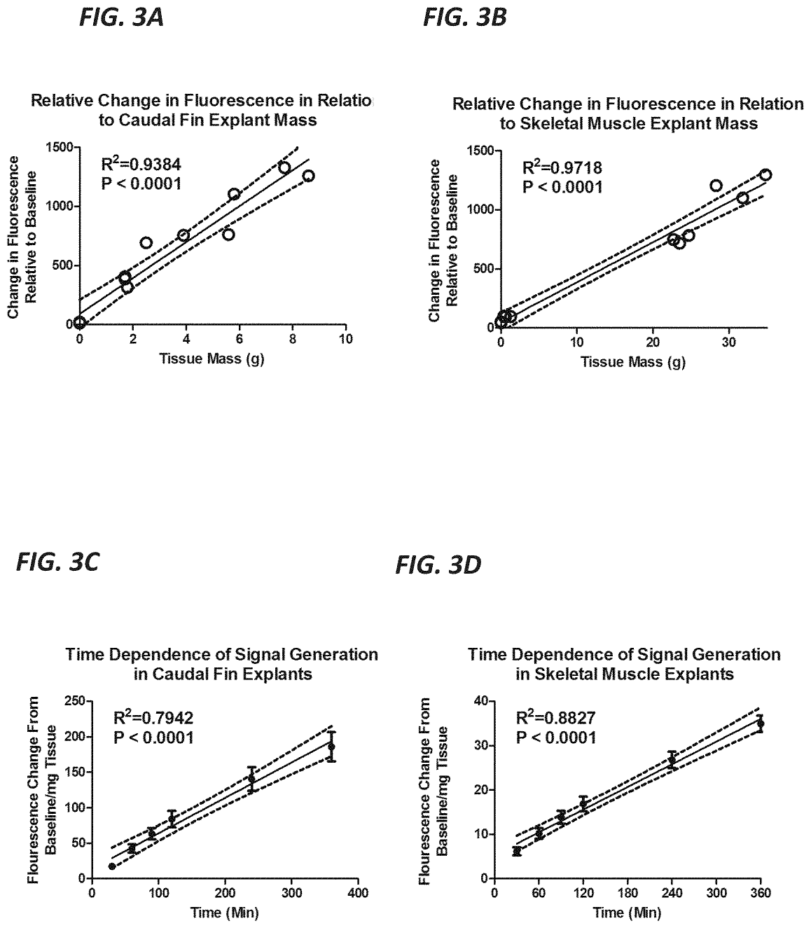

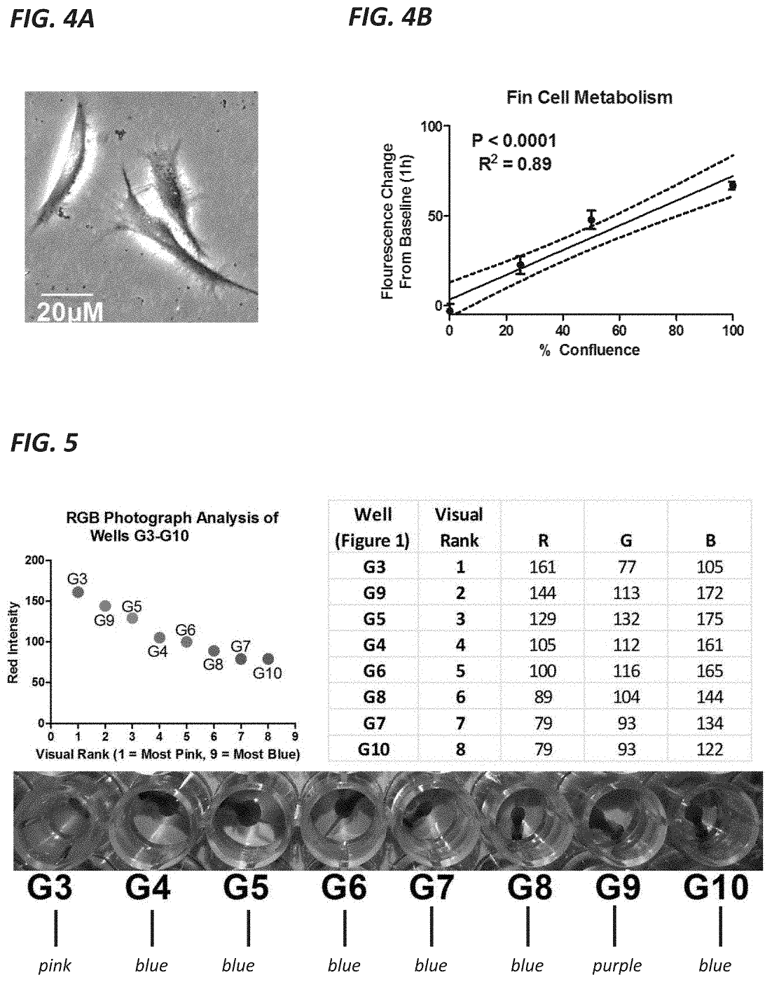

FIG. 1 shows that embryonic fish metabolic rate may be used to predict future growth. Further, if fish (e.g., tilapia) are segregated by metabolic rate, fish with a metabolic rate in the highest quartile grow faster than fish with a metabolic rate in the lowest quartile. FIG. 1 also suggests that high metabolic rate fish maintain a growth advantage for at least 8 months. In some embodiments, the present invention can be used to predict growth at 3 months, 4 months, 5 months, 6 months, 7 months, 8 months, 9 months, etc. In some embodiments, the present invention can be used to predict growth of the animal, e.g., fish, at harvest size/time. In some embodiments, the present invention can be used to predict growth of the animal, e.g., fish, at a time after the maternal effect time period has passed.

Regarding FIG. 1, studies on growth were conducted in three cohorts of high and low metabolic rate fish (n=3). Each cohort included measurement of metabolic rate in at least 6,000 embryonic fish so that each metabolic rate group included 1500 fish/experimental unit (n). Because these fish were fed identically, this increased growth is also indicative of improved feed conversion. Importantly, body length of these embryonic fish does not differ on the day and time that the AlamarBlue assay was completed (see FIG. 2). Thus, fish that are larger at the time were not selected for when the assay was performed.

An example of a method for measuring metabolic rate (e.g., metabolic rate assay) is as follows: Embryonic tilapia are rinsed 3 times in sterile 28° C. fish water. Using a disposable plastic pipette, embryonic fish are individually transferred into wells of a 96-well plate. Once plated, 96-well plates containing fish are put into an incubator to maintain a water temperature of 28° C. Upon completion of plating, all water is removed from the well and is replaced with 300 μl sterile filtered assay medium (Fish system water including, 4 mM NaHCO3, 0.1% DMSO, and 0.16% AlamarBlue (Cat. #Y00010; Thermo Fisher Scientific Inc.; Waltham, Mass.)). Fluorescence is determined using a fluorescent plate reader (excitation wavelength 530 and emission wavelength 590) at the beginning of the assay to establish a baseline for each embryo and at the end of the assay (e.g., at 16 h). A large change in fluorescence is indicative of robust NADH₂ production and a high metabolic rate, while a small change in fluorescence is indicative of a low metabolic rate.

The methods and systems of the present invention are not limited to the aforementioned example. Note that it may be possible for a single investigator to perform the metabolic rate assay on approximately 2,500 embryonic fish/8 hr day.

Although selection of broodstock as embryos may work well in captive bred fish with short generation intervals, application in either wild caught brood stock or slow maturing fish species requires the ability to assess the genetic potential for growth of adult fish. Preliminary studies establish that the methods and systems of the present invention (e.g., redox assay using AlamarBlue) may be used to assess the metabolic rate of skeletal muscle or caudal fin clip explants. FIG. 3A and FIG. 3B shows that within samples collected from the same fish, the change in fluorescence is directly related to the size of the fin or skeletal muscle explant. Moreover, like previously observed in embryonic fish, explants increase the signal generated with time (see FIG. 3C, FIG. 3D).

Epithelial cells from fin explants and satellite cells from skeletal muscle have been isolated. FIG. 4A shows the satellite cells from skeletal muscle can differentiate into proliferating myoblasts. FIG. 4B shows that AlamarBlue can measure changes in fish fin cell density within a well. Without wishing to limit the present invention to any theory or mechanism, it is believed that in vitro metabolic rate of skeletal muscle satellite cell derived myoblasts and caudal fin epithelial cells expressed as change in fluorescence per μg DNA may help segregate fish with high and low metabolic rates.

As previously discussed, the present invention features methods for predicting growth in an organism (e.g., a plant, aquatic animal, e.g., fish, embryonic fish, adult fish, etc.). In some embodiments, the method comprises measuring the metabolic rate in a cell sample of the organism. This may be done by performing a colorimetric/fluorescent redox assay comprising introducing a redox indicator (e.g., resazurin) to the cell sample at time T₀, measuring a fluorescence value at time T₀, and measuring a fluorescence value at time T₁. The change in fluorescence between T₁ and T₀ can be calculated to determine the metabolic rate. A high metabolic rate is indicative of a high growth rate, and a low metabolic rate is indicative of a low growth rate.

The redox assay may be performed over a period of time such as 2 hours, 4 hours, 6 hours, 8 hours, 10 hours, 12 hours, 14 hours, 16 hours, 20 hours, 24 hours, etc. As such, in some embodiments, T₁ is equal to T₀ plus 8 hours, T₀ plus 16 hours, T₀ plus 24 hours, etc. The present invention is not limited to these times.

Fluorescence may be determined in a variety of ways. For example, in some embodiments, fluorescence is calculated using a fluorescent plate reader. In some embodiments, absorbance/fluorescence is calculated using a digital photograph of the cell sample. In some embodiments, the red, green, and blue color intensity is quantitated. The intensities may be correlated with fluorescence to determine a quantitative measurement of metabolic rate.

The present invention also features a method for broodstock selection. A sample (e.g., cell sample) may be obtained from each fish (e.g., fish embryos, adult fish, etc.). In some embodiments, the method comprises measuring the metabolic rate in each tissue sample and comparing the metabolic rates of each fish in the pool. Particular fish may be selected based on their metabolic rate. For example, in some embodiments, fish that have a metabolic rate that is in the top 50%, top 40%, top 30%, top 20%, top 10%, etc. may be selected.

The present invention also features selection based on metabolic rate through computer aided red-green-blue (RGB) analysis of digital photographs (see FIG. 5.)

Because selection for the genetic potential for growth has been limited by 1) interactions between aggression and growth, 2) inability to select in wild-caught brood stock, and 3) the long generation interval in slow maturing species, the methods of the present invention may allow for selection of genetically superior brood stock, which may have a positive impact on profitability of the aquaculturalist. FIG. 5 shows that metabolic rate has a distribution with a right extending tail; thus, it may be possible to impose a large selection differential to maximize the response to selection based on metabolic rate. In those industries that are dependent on wild caught brood stock, the methods of the present invention may be used first to allow for selection. In late maturing species (e.g., sturgeon), by increasing growth it may be possible to shorten the duration to maturation.

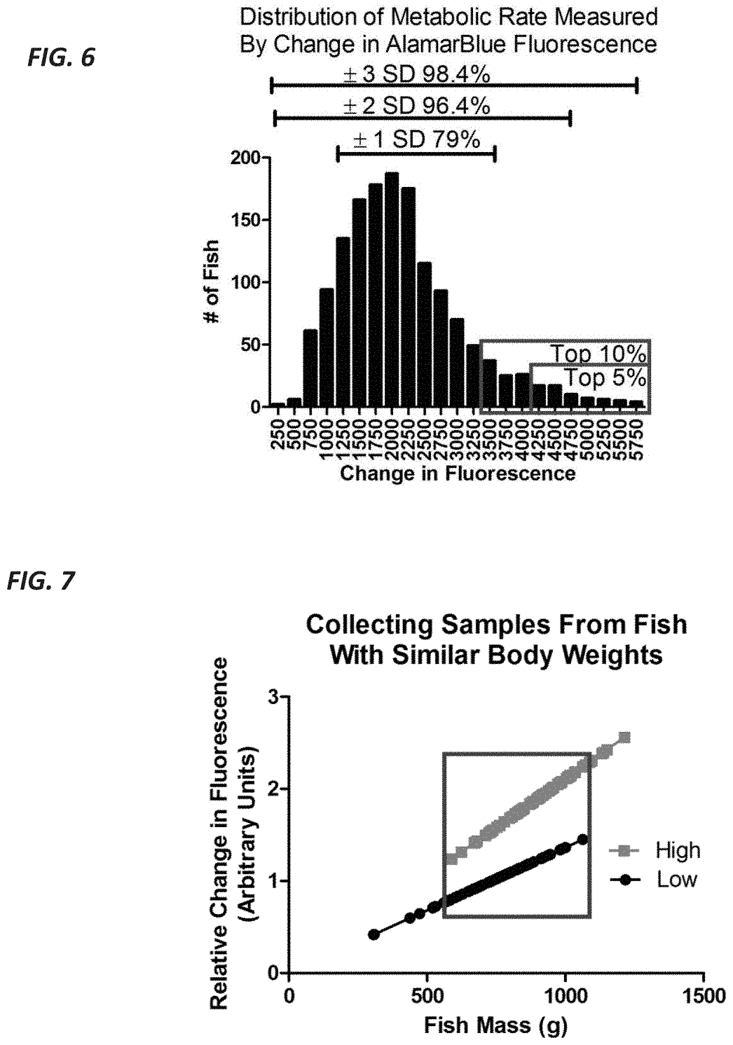

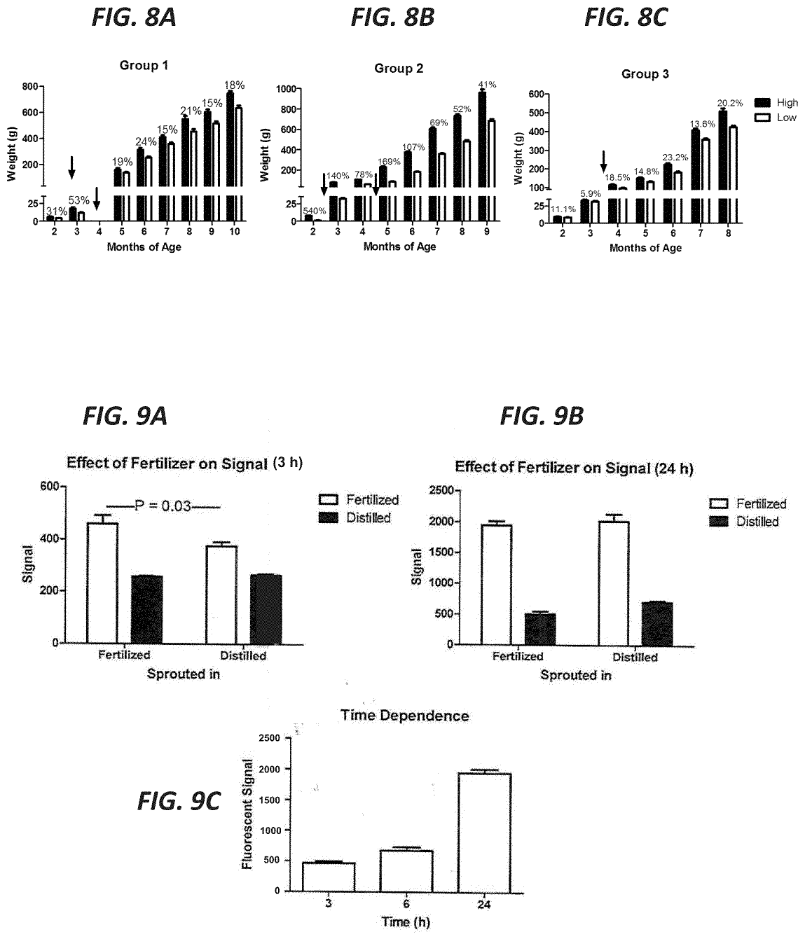

FIG. 6 shows a histogram displaying the frequency distribution of metabolic rates measured by change in fluorescence. Note the long right tail composed of fish that have a high selective differential from the mean. FIG. 7 shows comparisons between high and low metabolic rate fish made at identical weights. Slope within the metabolic rate group will determine the relationship between current body weight and explant metabolic rate. Body weight data is from fish that are already selected; thus, selection of fish with similar masses in each group may be feasible. FIG. 8A-8C show the growth advantage in tilapia that had a high metabolic rate (top quartile) as embryos relative to those embryos that have a low metabolic rate (bottom quartile). The percent increase in growth attributable to the high metabolic rate is shown at each time point for each group. At all time points significant differences existed.

As previously discussed and as shown in FIG. 9A-9C, the present invention may also be used in plants. For example, the present invention may be used to test soil, water, and/or fertilizers. In some embodiments, the plants with the best genetics for growth may be selected. In some embodiments, water quality or soil quality is assessed. In some embodiments, the ability of different fertilizers to enhance growth is assessed.

### Monitoring NADH₂ Production By Adult Cells

The following is an example of the use of the methods and systems of the present invention. The present invention is not limited to the details set forth below.

Below describes assessing metabolic rate in adult fish, e.g., assessing the metabolic rate of tissue explants/cells collected from adult fish that were segregated by metabolic rate as embryos. This may be used as a means of selecting for captive bred species. For example, the present invention may be used to assess the ability to ascertain genetic potential for growth of adult fish by measuring metabolic rate using minimally invasive, non-lethal techniques to collect fin and skeletal muscle samples. Without wishing to limit the present invention to any theory or mechanism, it is believed that the methods and systems of the present invention may be used to evaluate wild caught and slow maturing brood stock. For example, it is believed that adult cells/explants from fish that had a high metabolic rate as embryos will have a higher ex vivo metabolic rate than cells/explant from tilapia that had a low metabolic rate as embryos.

Fin Sampling: Fish will be weighed then anesthetized in a solution of tricaine methane sulfonate (MS222, 100 mg/L). Mucus will be wiped from the caudal fin. Sterile scissors or a tissue punch will be used to remove a piece of fin ray from between the bones of the caudal fin. Bacterial contamination will be limited by rinsing 3 times for 5 minutes in L-15 media with Gentamycin (100 μg/ml), and fungizone (2.5 μg/ml).

Explant metabolic rate: Tissue is plated in 300 μl assay media (L-15 media without phenol red supplemented with 25 mM HEPES, 5 mM NaHCO3, Penicillin-streptomycin (50 I.U./ml), 0.1% DMSO and 0.16% AlamarBlue). Fluorescence is measured at time 0 on a fluorescent plate reader set to excite at 530 nM and measure emission at 590 nM. Explants are incubated in a normal air incubator at 28° C. Fluorescence will again be measured at 1, 2, 3, 4, 6, 12, and 24 h to measure change across time. At 24 h the tissue will be collected, weighed, and homogenized in lysis buffer (0.1 M phosphate buffered saline with 0.1% Triton X-100, PBST) for analysis of DNA using the Quant-iT PicoGreen dsDNA assay kit (Life Technologies, Inc.) to correct all samples for the number of cells. Fluorescence data will be corrected by either tissue weight or total DNA and expressed as change in fluorescence per mg tissue or μg DNA.

Fin cell isolation will be performed as previously described. Upon reaching confluence in a 3.5 cm petri dish, cells will be plated to confluence in a 96-well plate and the AlamarBlue assay will be performed as described for explants and corrected for total DNA within the well.

Skeletal Muscle Sampling and Explant/Cell Isolation: Skeletal muscle samples will be collected by needle (14G) biopsy in the anesthetized fish from which fin samples were collected. 2-5 mg skeletal muscle explants will be assayed in triplicate to assess metabolic rate by explant. Changes in fluorescence will be corrected for sample DNA.

Skeletal muscle stem cells (satellite cells) will be isolated from the remainder of the biopsy and grown to confluence. Upon reaching confluence satellite derived proliferating myoblasts will be plated into a 96-well plate and the AlamarBlue assay will be performed as described for fin cells. Alternatively, myoblasts may be stimulated to form nascent muscle fibers so that metabolic rate can be measured on the differentiated cell type.

Without wishing to limit the present invention to any theory or mechanism, it is believed that samples collected from fish that were determined to have a low metabolic rate as embryos will maintain a lower metabolic rate than samples collected from fish that had a high metabolic rate as embryos. FIG. 3 and FIG. 4B show that metabolic rate in explants and isolated cells can be accurately measured. It is believed that the metabolic rate from both skeletal muscle and fin samples can be accurately assessed. And, it is believed that the metabolic rate assessed from skeletal muscle samples will correlate with metabolic rate assessed from fin samples.

Data may be analyzed in SAS. The effect of embryo metabolic rate (high or low), adult body weight, and their interaction on metabolic rate from explants or cells will be assessed for each tissue type using a mixed model two-way ANOVA in SAS (SAS Inc., Cary, N.C.). Correlation analysis will be used to assess relationships between metabolic rate assessed by tissue type and sample type (cells or explants; Proc Corr). Regression analyses will be performed to quantitate this effect as needed.

The present invention may also be used to assess selection of broodstock based on metabolic rate of tissue samples, e.g., metabolic rate of explants from adult fish may be indicative of offspring metabolic rate. Future work may focus on this application in wild-caught brood stock.

Methods: Broodstock rearing and selection: 300 fish will be grown in a starter tank and moved as needed to maximally encourage growth and development. Feed will be provided 3 times daily to satiation. At 4 months of age, skeletal muscle or fin biopsies will be collected for metabolic rate determination as described and validated in Experiment 2. Fish will be tagged for identification and upon determination of metabolic rate, fish in the top and bottom 10% will be isolated and moved to other facilities.

Breeding: Brood stock in the high and low metabolic rate groups will be divided into five tanks with 6 brood stock in each tank (n=5). The mouths of female fish will be checked every week for embryonic fish. Embryos will be collected and assayed for metabolic rate as previously described. To prevent continuously sampling from the same female fish, a female that provides a clutch will be immediately removed from the study. Studies will continue until at least 2 clutches have been collected and analyzed from each tank.

A one-way ANOVA will be used to assess the effect of explant/cell metabolic rate on embryo metabolic rate. Tank will serve as the experimental unit (n=5). Accordingly, the single measurement of each embryo will be nested within brood and brood nested within tank. Power calculation mirrors that from experiment 1.

Without wishing to limit the present invention to any theory or mechanism, it is believed that the metabolic rate of explants from adult fish will be positively related to the offspring metabolic rate. This result would indicate that brood stock can be selected based on metabolic rate of adult tissue samples.

### Field Application

The Field Application is an example of the use of the methods and systems of the present invention. The present invention is not limited to the details set forth in the Field Application.

The following describes procedures that allow for field application of the methods and systems of the present invention, e.g., assessing results of methods of the present invention using a controlled digital photograph, eliminating the requirement for a fluorescent spectrophotometer.

First, the quantitative assessment of color change from digital photographs will be compared to that obtained using a fluorescent plate reader, e.g., quantitated RGB color will be correlated with fluorescent signal measured on a plate reader. Without wishing to limit the present invention to any theory or mechanism, it is believed that the use of digital photographs may be an adequate substitute for a fluorescent plate reader, allowing the methods of the present invention to be performed in the field.

Methods: Plates are digitally captured using a high resolution digital camera. In all pictures, a standard white background is used and a color reference chart is included to correct for potential differences in lighting. Using Adobe Photoshop eyedropper tool, the red, green, and blue color within each well will be quantitated (see FIG. 5, preliminary data). The red, green, and blue color intensity, differences between intensity of different colors (e.g. Red-Green), and ratios of color intensity (e.g. Red/Blue) are correlated with fluorescence to identify the color spectrum quantitation that best allows for quantitative analysis of metabolic rate from a color photo. Data may first be analyzed through XY plots using GraphPad Prism version 5.00 for Windows, GraphPad Software, San Diego Calif. USA, www.graphpad.com to visually understand the relationship between fluorescence and each variable of color (intensity of a single color, differences, ratios). It is possible that linear relationships will be found, and thus Proc Corr in SAS (SAS Institute, Cary, N.C.) will then be performed to identify the factor with the greatest correlation with change in fluorescence. Proc Reg in SAS may be used to quantitate the relationship between fluorescence and quantitated RGB spectrum. If fluorescence and RGB values are not linearly related the data may be transformed (or a non-linear regression analysis may be performed).

Next, the possibility of shipping explants for analysis in a central lab will be assessed. Without wishing to limit the present invention to any theory or mechanism, it is believed that explants may retain their metabolic rate (e.g., fluorescence per mg tissue) for a period of time, e.g., 12 hr or more, after collection. Maintaining explants at 4° C. may extend viability.

Methods: The differences in the timing of assay initiation and sample incubation temperature will be compared to assess the possibility that explants could be collected on farm and shipped to a commercial laboratory for analysis. The present invention establishes that embryonic fish and oysters can be collected and shipped to a central lab for analysis, thus it may be possible for explants as well.

Within a fish, the coefficient of variation in metabolic rate of skeletal muscle and fin explants is low (5.7 and 6.2%, respectively). By collecting multiple samples from the same individuals, it may be possible to perform comparisons to thoroughly analyze the effects of assay timing and incubation temperatures on explant viability. This study will be conducted testing 5 different times (0, 6, 12, 24, and 48 h) and 2 different incubation temperatures (4 or 22 degrees C.). Each condition will be run in triplicate within a fish. As such, 30 samples/fish are needed. To accommodate the need for this large number of samples/fish, samples will be collected immediately post-mortem from fish anesthetized in an ice water slurry and sacrificed by decapitation. Each sample will be placed in a capped culture tube containing 1 ml L-15 medium without phenol red supplemented with 25 mM HEPES, 5 mM NaHCO3, Gentamycin (100 μg/ml), and fungizone (2.5 μg/ml). Three samples from each fish will be exposed to each condition. Samples maintained at both room temperature and at 4° C. will be kept in Styrofoam shipping containers within the lab and samples will be removed at 6, 12, 24, and 48 hours of incubation. At the end of the incubation samples will be analyzed as previously described for metabolic rate. At the conclusion of the study, explants will be weighed and total DNA within the sample will be assessed. Fluorescence data will be corrected by either tissue weight or total DNA and expressed as change in fluorescence per mg tissue or μg DNA.

### Additional Experiments

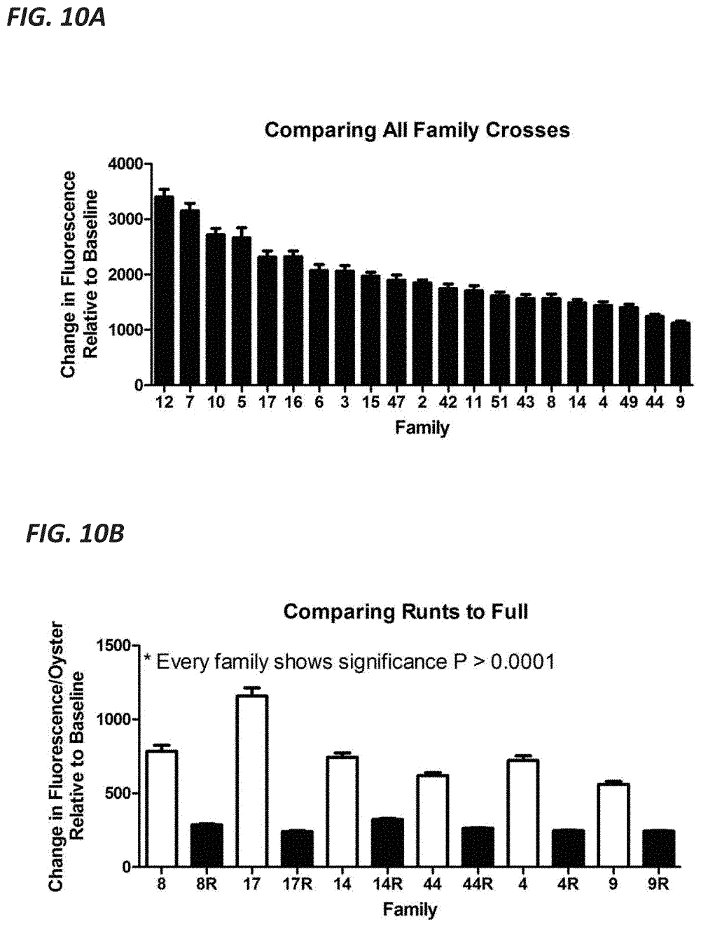

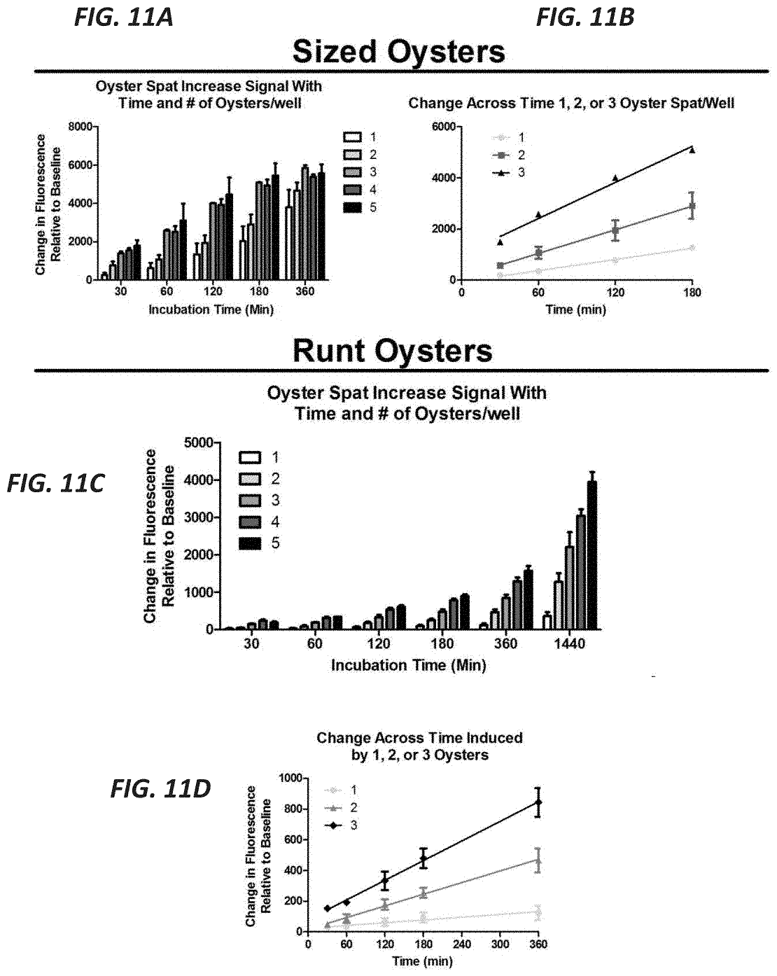

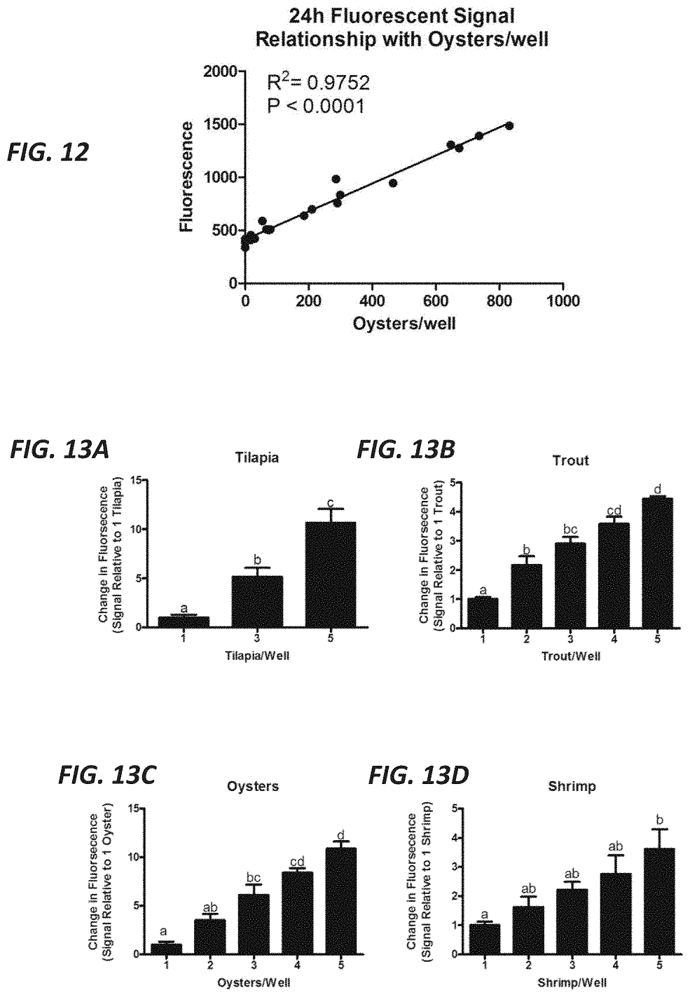

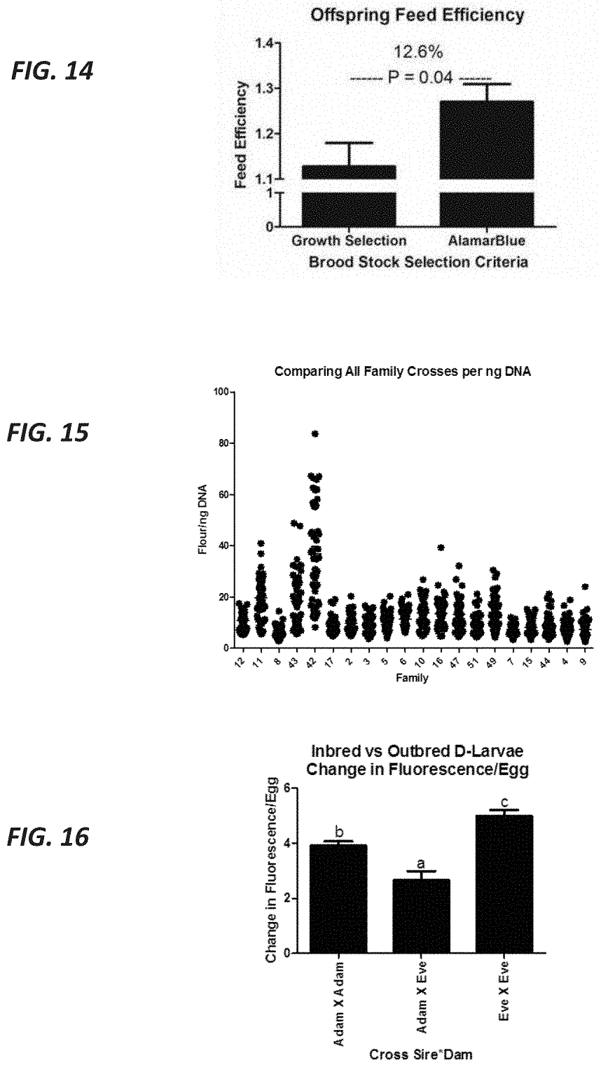

FIG. 10A-10B show representative results from Oyster Spat. The top figure shows that oyster spat derived from different families induced nearly 3 fold differences in AlamarBlue reduction. This suggests that significant variation in NADH₂ production exists between and within families. In the middle figure we can see that if oyster spat within a cross are size separated, those spat that grow more slowly (runts; R) induce less signal than oysters that crow more quickly. FIG. 11A-11D show that in both sized and runt oysters the signal generated increases with time and with the number of oysters within a well. Larger oysters (sized) generate signal more quickly and with fewer oysters than do runt oysters. FIG. 12 shows D-larvae (2-day post fertilization larval oysters) increase the signal generated in this AlamarBlue based assay linearly as the number of larvae within a well increases (R²=0.9752). FIG. 13A-13D show changes in fluorescence with various numbers of tilapia (FIG. 13A), trout (FIG. 13B), oysters (FIG. 13C), and shrimp (FIG. 13D). FIG. 13A-13D show that metabolic rate within a given volume increases as the number of individuals within that volume increases. FIG. 14 shows that tilapia broodstock selection based on metabolic rate, as measured by the AlamarBlue based assay, results in offspring with higher feed efficiency than offspring from Tilapia broodstock selection based on growth. FIG. 15 shows the variability in metabolic rate within a family. This proposes that broodstock selection based on family selection will not improve genetics for growth nearly as rapidly as selection based on the individual metabolic rate. FIG. 16 shows that slow growing inbred oyster lines (adam×adam) and (eve×eve) have higher metabolic rates than their outcrossed faster growing half-siblings (adam×eve).

### EXAMPLE 2

The present invention also describes methods and systems for preparing groups (e.g., creating groups, separating groups, etc.) of embryonic fish or juvenile aquatic organisms from a pool based on predicted metabolic rate relative to the other individuals in the pool. The method and systems herein may be used to create or prepare a group (or sequester) of individuals with highest growth potential or best feed efficiency.

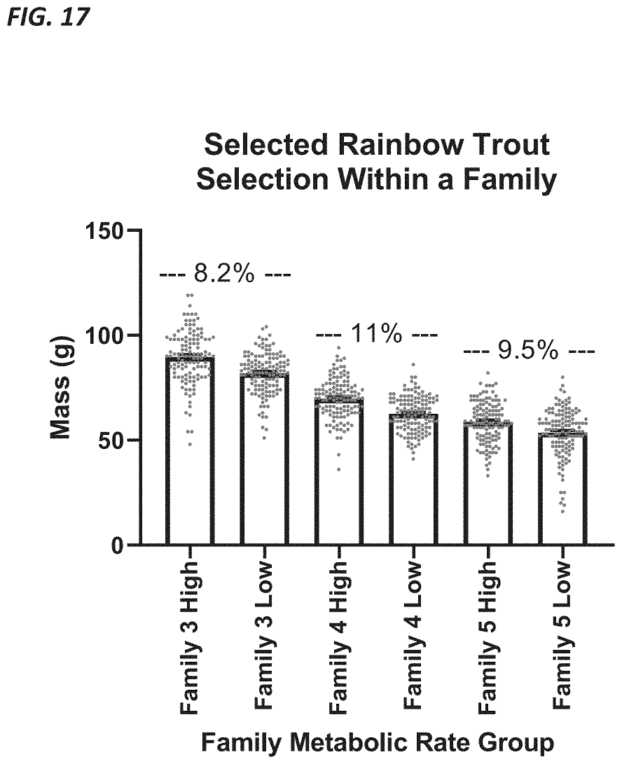

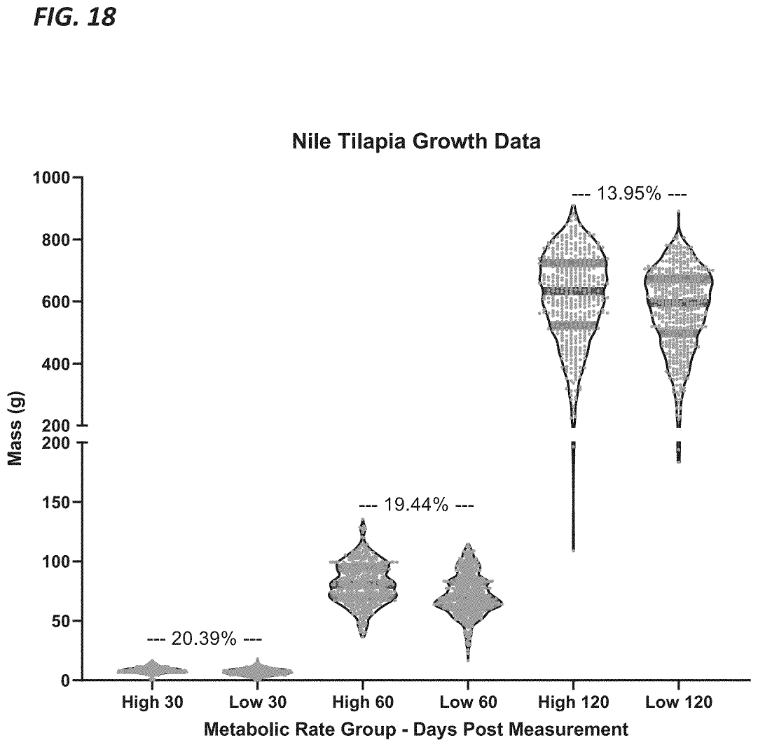

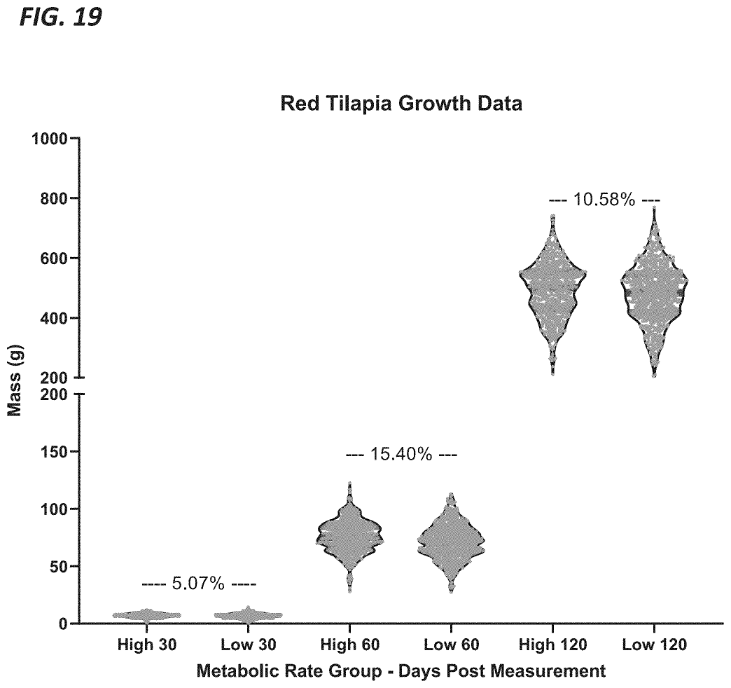

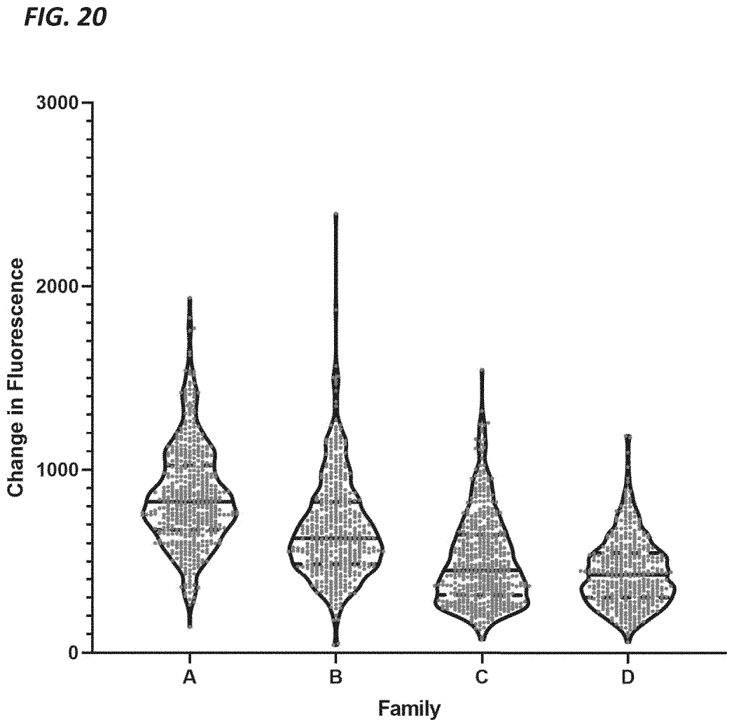

FIG. 17, FIG. 18, FIG. 19, and FIG. 20 each show experimental evidence that high metabolic rate aquatic organisms become those from a pool (e.g., family) that grow more quickly than the low metabolic rate organisms from the pool. For example, within a dutch, high metabolic rate rainbow trout eggs (FIG. 17), Nile tilapia embryos (FIG. 18), and Red tilapia (FIG. 19) become fish that grow more quickly than the low metabolic rate individuals in the dutch. FIG. 20 shows that Atlantic salmon eggs from growth-selected fish (A and C, e.g., selected based on predicted growth) create more signal than salmon eggs from brood that were selected based on immune function (B and D). Older eggs (A and B) create more signal than younger eggs (C and D).

The present invention provides automated systems and methods for preparing (e.g., creating, generating, etc.) groups of embryonic fish or juvenile aquatic organisms from a pool based on predicted growth potential or feed efficiency.

For example, the present invention provides computer implemented methods, wherein the method comprises measuring whole-body metabolic rate of an individual in the pool of embryonic fish or juvenile aquatic organisms (e.g., detecting a colorimetric/fluorescent redox indicator) and moving the individual to a particular container if the whole-body metabolic rate is above a precalculated threshold of fluorescence that would be indicative of high metabolic rate (relative to the pool). For example, the highest metabolic rate individuals may be placed in a first container, and the remaining individuals may be placed in one or more different containers, e.g., depending on the relative metabolic rate of those individuals. In some embodiments, the method features the use of two containers. In some embodiments, the method features the use of three containers. In some embodiments, the method features the use of four or more containers. The steps may be repeated for all individuals in the pool.

The present invention also provides systems, e.g., computer implemented systems, wherein the system comprises a detector for detecting a colorimetric/fluorescent redox indicator in an individual of the pool (e.g., for measuring a fluorescence value for the individual); a sorting component for moving an individual of the pool to one of two or more containers after detection of the redox indicator; and a microprocessor operatively connected to the detector and the sorting component. The microprocessor is configured to receive input signals from the detector as fluorescence values of individuals of the pool and send output signals to the sorting component to move the individuals to one of two or more containers (e.g., two containers, three containers, four containers, more than four containers, etc.). In some embodiments, the detector is a visual detector, a photodetector, a photodiode detector, a fluorescent detector, a camera, or the like. The present invention is not limited to any particular detector.

In some embodiments, the microprocessor is configured to calculate a precalculated threshold of fluorescence based on input from the detector, e.g., a collection of fluorescence values obtained from the detector. The precalculated threshold of fluorescence is a minimum fluorescence value indicative of a predicted high metabolic rate individual. For example, the threshold refers to the lowest fluorescence value that would classify that individual as one that would fall into the highest X % of metabolic rates (e.g., highest 10%, highest 20, 25%, 30%, 40%, 50%, etc.). In some embodiments, the microprocessor is configured to send an output signal to the sorting component to move the individual to a first container if the individual has a fluorescence value higher than the precalculated threshold of fluorescence; or send an output signal to the sorting component to move the individual to a container different from the first container (e.g., one of two other containers, one of three other containers, etc.) if the individual has a fluorescence value below the precalculated threshold of fluorescence.

The present invention also provides a computer implemented method, wherein the method comprises introducing to the pool of embryonic fish or juvenile aquatic organisms a colorimetric/fluorescent redox indicator, wherein fluorescence of the colorimetric/fluorescent redox indicator is for determining relative whole-body metabolic rate. In some embodiments, the method comprises introducing the pool of embryonic fish or juvenile aquatic organisms to an automated system, wherein the automated system sequentially detects fluorescence values of n embryonic fish or juvenile aquatic organisms (E₁-Eₙ) and calculates a threshold fluorescence value V corresponding to a minimum fluorescence value within a X % highest fluorescence values detected. The values for n and X may be pre-programmed by a user. The method may further comprise sequentially detecting a fluorescence value of an additional embryonic fish or juvenile aquatic organism (Eₙ₊₁) and moving Eₙ₊₁ to a first container if the fluorescence value is greater than V or a container different from the first container if the fluorescence value is less than V.

In some embodiments, the method further comprises recalculating a new V based on fluorescence values of the previous n embryonic fish or juvenile aquatic organisms or a portion thereof. In some embodiments, the method further comprises detecting a fluorescence value of a new embryonic fish or juvenile aquatic organism and moving the new embryonic fish or juvenile aquatic organism to a first container if the fluorescence value is greater than the new V or container different from the first container if the fluorescence value is less than the new V. In some embodiments, the method further comprises repeating the steps: recalculating a new V based on fluorescence values of the previous n embryonic fish or juvenile aquatic organisms (or a portion thereof); and detecting a fluorescence value of a new embryonic fish or juvenile aquatic organism and moving the new embryonic fish or juvenile aquatic organism to a first container if the fluorescence value is greater than the new V or a second container if the fluorescence value is less than the new V.

In some embodiments, n is 500. In some embodiments, n is from 10 to 10,000.

For any of the embodiments herein, the embryonic fish or juvenile aquatic organisms are aquaculturally farmed organisms. The embryonic fish may be salmon, trout, zebrafish, sea bass, tilapia, catfish, sole, cobia, or the like. The present invention is not limited to any particular embryonic fish and may include any aquaculturally farmed fish. In some embodiments, the juvenile aquatic organisms are shrimp, lobster, prawn, or tiger prawn. The present invention is not limited to any particular juvenile aquatic organism and may include any aquaculturally farmed organism.

The present invention also provides a computer implemented sorting system, wherein the system comprises a detector for detecting a colorimetric/fluorescent redox indicator in an individual of the pool so as to measure a fluorescence value for the individual; a sorting component for moving an individual of the pool to one container out of c containers after detection of the redox indicator, wherein c is at least two; and a microprocessor operatively connected to the detector and the sorting component. The microprocessor receives input signals from the detector as fluorescence values of individuals of the pool and sends output signals to the sorting component to move the individuals to one of c containers.

In some embodiments, the microprocessor is configured to receive and store fluorescence values from the detector as the detector sequentially detects light emitted from n embryonic fish or juvenile aquatic organisms (E₁ through Eₙ), wherein n is pre programmed by a user, wherein n is at least 10. In some embodiments, the microprocessor is configured to organize a distribution of fluorescence values of all (or a portion) of the n detected embryonic fish or juvenile aquatic organisms. In some embodiments, the microprocessor is configured to calculate V, wherein V is a minimum fluorescence value corresponding to the X % highest fluorescence values in the distribution, wherein X is pre-programmed by a user. In some embodiments, the microprocessor is configured to receive and store a fluorescence value from the detector when the detector detects light emitted from a new embryonic fish or juvenile aquatic organism (Eₙ₊₁). In some embodiments, the microprocessor is configured to (i) send a first output signal to the sorting component to direct Eₙ₊₁ to a first container if the fluorescence value of Eₙ₊₁ is above V, or (ii) send a second output signal to the sorting mechanism to direct Eₙ₊₁ to a container different from the first container if the fluorescence value of Eₙ₊₁ is below V.

For example, the system may start by detecting 500 embryonic fish (fluorescence values of the embryonic fish) and using said data create a distribution of the fluorescence values. Further, the system may calculate V, the minimum fluorescence value needed to be considered a fluorescence value in the top X % (e.g., 10%, 15%, 20% 25%, 30%, 35%) of highest whole-body metabolic rates. After having calculated V, the system reads a new embryonic fish. If the new embryonic fish has a fluorescence value greater than V, it is sorted into one particular container. If the fluorescence value is less than V, it is sorted into one (or one of two or more) different containers. Next, the system recalculates V based on the previous n embryonic fish or other organism (e.g., juvenile aquatic organism) or a portion thereof. The system can repeat the aforementioned steps, e.g., for 1,000 embryonic fish or other organism (e.g., juvenile aquatic organism), 5,000 embryonic fish or other organism (e.g., juvenile aquatic organism), 10,000 embryonic fish or other organism (e.g., juvenile aquatic organism), 100,000 embryonic fish or other organism (e.g., juvenile aquatic organism), etc. Thus, V is recalculated after each new embryo.

As a non-limiting example, the system initially reads/detects a certain number (n) embryos, e.g., 500 embryos. Based on the initial n (e.g., 500) readings/values (e.g., “fluorescence values,” “detection values”), the system can calculate the fluorescence values (or detection values) that are the cut-offs (or “precalculated thresholds”) for particular percentiles of relative whole-body metabolic rate. For example, a user may pre-program the system to sort individuals in the 10^{th} percentile and above (e.g., the top 10%) into one container, individuals in the 50^{th} to 100^{th} percentile (e.g., the bottom 50%) into a second container, and remaining individuals (between the 10^{th} percentile and 50^{th} percentile) into a third container. Thus, the system calculates the cut-off fluorescence value(s) that would lead to an individual being in the top 10%, bottom 50%, or in between of relative whole-body metabolic rates. After the initial embryos are read/detected and the system calculates the cut-offs (or precalculated thresholds), the system continues to sequentially read/detect embryos in the pool. When the next embryo is detected (embryo n+1, Eₙ₊₁), the system reads the fluorescence value (or detection value) and sorts the embryo based on whether the embryo fits into the top 10%, bottom 50%, or in between. Next, the system uses the fluorescence value of embryo n+1 (Eₙ₊₁) it has just detected and recalculates the cut-off values (for those same percentiles) based on the previous n embryos (or a portion thereof), e.g., embryo n+1 and the n−1 previous embryos or a portion thereof. This pattern repeats as each additional embryo is detected/read (or as nearly each additional embryo is detected). This continuous or near continuous recalculation of the cut-off values can help to reduce the impact that diffusion of the indicator (or other factors that cause reduction in signals over time) may have on the fluorescence value of the subsequent individual. For example, there would naturally be a general decrease in signal over time for the individuals in the pool, so the cut-off values for the initial embryos detected would not necessarily be applicable to the individuals detected at the end of the process.

As previously discussed, the microprocessor is configured to recalculate a new V based on fluorescence values of the previous n embryonic fish or juvenile aquatic organisms (or a portion thereof). In some embodiments, the number of containers (e.g., “c”) is at least 3. In some embodiments, the number of containers (e.g., “c”) is from 2 to 10. In some embodiments, n is 500. In some embodiments, n is from 10 to 10,000.

The present invention is not limited to the exact specific calculations described herein. Additional embodiments that provide an equivalent to the described calculations are within the scope of the invention. Thus, for example, the present invention includes embodiments wherein the system recalculates V based on not 100% of the previous n embryos (or juvenile aquatic organisms or mollusks, etc.) but a portion thereof, e.g., percentage (e.g., 90%, 80%, 70%, 60%, 50%) of the previous n embryos (or juvenile aquatic organisms or mollusks, etc.).

For any of the embodiments herein, the methods may comprise preparing the groups (e.g., the separated groups) of organisms for distribution or sale. The methods may comprise preparing the groups of organisms (the separated groups) for growth.

The present invention describes the use of indicators for determining relative metabolic rate of the embryonic fish or juvenile aquatic organisms. In certain embodiments, the indicators are for measuring the flux of one or more metabolites through metabolic oxidation, e.g., indicators of the production of NADH₂, which is a product of metabolism. The methods and systems of the present invention may detect and/or monitor NADH₂ using a redox indicator. Non-limiting examples of indicators include resazurin/resorufin or other appropriate indicators (e.g., NADH₂ indicators) such as but not limited to tetrazolium dyes, e.g., MTT, XTT, MTS, WST. In certain embodiments, the redox indicator is reduced by NADH₂, resulting in a colorimetric and/or fluorescent shift in solution. The indicator may be used to determine a relative metabolic rate of an individual, e.g., relative to a portion of the individuals in the pool.

### Mollusks

The present invention also features methods and systems for generating (e.g., preparing) groups or selections of mollusks (e.g., oysters) from a pool. As described above, the mollusks may be separated into different groups based on relative metabolic rate. In a hypertonic solution, the mollusks with the lower whole-body metabolic rate are predicted to grow faster than the mollusks with the higher whole-body metabolic rate. Thus, the methods encompass calculating a threshold wherein the individuals below the threshold are moved to the first container and the individuals above the threshold are moved to a different container.

For example, the present invention provides an automated computer implemented method for preparing groups of mollusks from a pool based on predicted growth potential or feed efficiency. In some embodiments, the method comprises measuring whole-body metabolic rate of an individual in the pool of mollusks, wherein metabolic rate is measured by detection of a colorimetric/fluorescent redox indicator; and moving the individual to a first container if the whole-body metabolic rate is below a precalculated threshold of fluorescence or moving the individual to a container different from the first container if the whole-body metabolic rate is above a precalculated threshold of fluorescence. The method may comprise repeating the above steps for all of the individuals in the pool.

The present invention also features an automated computer implemented system for preparing groups of mollusks from a pool based on predicted growth potential or feed efficiency. In some embodiments, the system comprises a detector for detecting a colorimetric/fluorescent redox indicator in an individual of the pool so as to measure a fluorescence value for the individual; a sorting component for moving an individual of the pool to one of two or more containers after detection of the redox indicator; and a microprocessor operatively connected to the detector and the sorting component, wherein the microprocessor receives input signals from the detector as fluorescence values of individuals of the pool and sends output signals to the sorting component to move the individuals to one of two or more containers. The microprocessor is configured to: calculate a precalculated threshold of fluorescence based on input from the detector, the precalculated threshold of fluorescence is a maximum fluorescence value indicative of a predicted low metabolic rate individual; and send an output signal to the sorting component to move the individual to a first container if the individual has a fluorescence value below the precalculated threshold of fluorescence; or send an output signal to the sorting component to move the individual to a container different from the first container if the individual has a fluorescence value above the precalculated threshold of fluorescence.

The present invention also features a computer implemented method for preparing groups of mollusks from a pool based on predicted growth potential or feed efficiency. In some embodiments, the method comprises introducing to the pool of mollusks a colorimetric/fluorescent redox indicator, wherein fluorescence of the colorimetric/fluorescent redox indicator is for determining relative whole-body metabolic rate; introducing the pool of mollusks to an automated system, wherein the automated system sequentially detects fluorescence values of n mollusks (M₁-Mₙ) and calculates a threshold fluorescence value V corresponding to a maximum fluorescence value within a X % lowest fluorescence values detected, wherein n and X are pre-programmed by a user; and sequentially detecting a fluorescence value of an additional mollusk (Mₙ₊₁) and moving Mₙ₊₁ to a first container if the fluorescence value is less than V or a container different from the first container if the fluorescence value is greater than V.

In some embodiments, the method further comprises recalculating a new V based on fluorescence values of the previous n mollusks (or a portion thereof). In some embodiments, the method further comprises detecting a fluorescence value of a new mollusk and moving the new mollusk to a first container if the fluorescence value is less than the new V or container different from the first container if the fluorescence value is greater than the new V. In some embodiments, the method repeats the steps: recalculating a new V based on fluorescence values of the previous n mollusks or a portion thereof; and detecting a fluorescence value of a new mollusk and moving the new mollusk to a first container if the fluorescence value is less than the new V or a second container if the fluorescence value is greater than the new V.

In some embodiments, the detector is a visual detector, a photodetector, a photodiode detector, a fluorescent detector, or a camera.

In some embodiments, the system (e.g., computer implemented system) for preparing groups of mollusks from a pool into groups based on predicted growth potential or feed efficiency comprises a detector for detecting a colorimetric/fluorescent redox indicator in an individual of the pool so as to measure a fluorescence value for the individual; a sorting component for moving an individual of the pool to one container out of c containers after detection of the redox indicator (e.g., two containers, at least two containers, three containers, etc.); and a microprocessor operatively connected to the detector and the sorting component. The microprocessor receives input signals from the detector as fluorescence values of individuals of the pool and sends output signals to the sorting component to move the individuals to one of c containers. The microprocessor is configured to receive and store fluorescence values from the detector as the detector sequentially detects light emitted from n mollusks (M₁ through Mₙ), wherein n is pre programmed by a user, wherein n is at least 10; organize a distribution of fluorescence values of all or a portion of the n detected mollusks; calculate V, wherein V is a maximum fluorescence value corresponding to the X % lowest fluorescence values in the distribution, wherein X is pre-programmed by a user; and receive and store a fluorescence value from the detector when the detector detects light emitted from a new mollusk (Mₙ₊₁) and (i) send a first output signal to the sorting component to direct Mₙ₊₁ to a first container if the fluorescence value of Mₙ₊₁ is below V, or (ii) send a second output signal to the sorting mechanism to direct Mₙ₊₁ to a container different from the first container if the fluorescence value of Mₙ₊₁ is above V. The microprocessor is configured to recalculate a new V based on fluorescence values of the previous n mollusks or a portion thereof.

In some embodiments, the number of containers (e.g., “c”) is at least 3. In some embodiments, the number of containers (e.g., “c”) is from 2 to 10. The present invention is not limited to one particular mollusk, e.g., oysters. In some embodiments, the mollusks are aquaculturally farmed. In some embodiments, n is 500. In some embodiments, n is from 10 to 10,000.

The disclosures of the following documents are incorporated in their entirety by reference herein: (1) Gjedrem, T., Aquaculture Research, 2000. 31(1): p. 25-33. (2) Conceicao, L. E. C., Y. Dersjant-Li, and J. A. J. Verreth, Aquaculture, 1998. 161(1-4): p. 95-106. (3) Gjerde, B., Aquaculture, 1986. 57(1-4): p. 37-55. (4) Huang, C. M. and I. C. Liao, Aquaculture, 1990. 85(1-4): p. 199-205. (5) Hulata, G., G. W. Wohlfarth, and A. Halevy, Aquaculture, 1986. 57(1-4): p. 177-184. (6) Tave, D. and R. O. Smitherman, Transactions of the American Fisheries Society, 1980. 109(4): p. 439-445. (7) Thodesen, J., et al., Aquaculture, 2011. 322: p. 51-64. (8) Gadagkar, S. R., Social behaviour and growth rate variation in cultivated tilapia (Oreochromis niloticus). 1997, Dalhousie University: Dalhousie University. (9) Koebele, B., Environmental Biology of Fishes, 1985. 12(3): p. 181-188. (10) Blanckenhorn, W. U., Ethology Ecology & Evolution, 1992. 4(3): p. 255-271. (11) Seiler, S. M. and E. R. Keeley, Animal Behaviour, 2007. 74(6): p. 1805-1812. (12) Grant, J. W. A., Canadian Journal of Fisheries and Aquatic Sciences, 1990. 47(5): p. 915-920. (13) Huntingford, F. A., et al., Journal of Fish Biology, 1990. 36(6): p. 877-881. (14) McCarthy, I. D., C. G. Carter, and D. F. Houlihan, Oncorhynchus mykiss (Walbaum). Journal of Fish Biology, 1992. 41(2): p. 257-263. (15) Allee, W. C., et al., Journal of Experimental Zoology, 1948. 108(1): p. 1-19. (16) Magnuson, J. J., Canadian Journal of Zoology, 1962. 40(2): p. 313-363. (17) Purdom, C. E., Variation in Fish, in Sea Fisheries Research, F. R. H. Jones, Editor. 1974, Elek Science: London. p. 347-355. (18) BROWN, M. E., Journal of Experimental Biology, 1946. 22(3-4): p. 118-129. (19) Wohlfarth, G. W., Shoot carp. Bamidgeh, 1977. 29(2): p. 35-56. (20) Clarke, A. and N. M. Johnston, Journal of Animal Ecology, 1999. 68(5): p. 893-905. (21) Miyashima, A., et al., Aquaculture Research, 2012. 43(5): p. 679-687, (22) Cook, J. T., A. M. Sutterlin, and M. A. McNiven, Aquaculture, 2000. 188(1-2): p. 47-63. (23) Livingston, R. J., Journal of the Marine Biological Association of the United Kingdom, 1968. 48: p. 485-497. (24) Renquist, B. J., et al., Zebrafish, 2013. 10(3): p. 343-52. (25) Williams, S. Y. and B. J. Renquist, Journal of Visualized Experiments, 2015. In Press. (26) Smith, R. W. and D. F. Houlihan, Journal of Comparative Physiology B, 1995. 165(2): p. 93-101. (27) Brand, M. D., et al., Evolution of energy metabolism. Proton permeability of the inner membrane of liver mitochondria is greater in a mammal than in a reptile. Biochem J, 1991. 275 (Pt 1): p. 81-6. (28) El-Greisy, Z. A. and A. E. El-Gamal, The Egyptian Journal of Aquatic Research, 2012. 38(1): p. 59-66. (29) Siraj, S. S., et al. International Symposium on Tilapia in Aquaculture. 1983. Nazareth, Isreal: Tel Aviv University. (30) Palada-de Vera, M. S. and A. E. Eknath. Proceedings of the Fourth International Symposium on Genetics in Aquaculture. 1993. Wuhan, Hubei Province, China: Elsevier. (31) Mauger, R. E., P. Y. Le Bail, and C. Labbe, Comparative Biochemistry and Physiology B-Biochemistry & Molecular Biology, 2006. 144(1): p. 29-37. (32) Vanmeter, D. E., Progressive Fish-Culturist, 1995. 57(2): p. 166-167.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A computer implemented sorting system for preparing groups of embryonic fish or juvenile aquatic organisms from a pool into groups based on predicted growth potential or feed efficiency, said system comprising:
a. a detector for detecting a colorimetric/fluorescent redox indicator in an individual of the pool so as to measure a fluorescence value for the individual; and
b. a sorting component for moving an individual of the pool to one container out of c containers after detection of the redox indicator, wherein c is at least two; and
c. a microprocessor operatively connected to the detector and the sorting component, wherein the microprocessor receives input signals from the detector as fluorescence values of individuals of the pool and sends output signals to the sorting component to move the individuals to one of c containers, wherein the microprocessor is configured to:
i. receive and store fluorescence values from the detector as the detector sequentially detects light emitted from n embryonic fish or juvenile aquatic organisms (E₁ through Eₙ), wherein n is pre programmed by a user, wherein n is at least 10;
ii. organize a distribution of fluorescence values of all or a portion of the n detected embryonic fish or juvenile aquatic organisms;
iii. calculate V, wherein V is a minimum fluorescence value corresponding to the X % highest fluorescence values in the distribution, wherein X is pre-programmed by a user;
iv. receive and store a fluorescence value from the detector when the detector detects light emitted from a new embryonic fish or juvenile aquatic organism (Eₙ₊₁) and (i) send a first output signal to the sorting component to direct Eₙ₊₁ to a first container if the fluorescence value of Eₙ₊₁ is above V, or (ii) send a second output signal to the sorting mechanism to direct Eₙ₊₁ to a container different from the first container if the fluorescence value of Eₙ₊₁ is below V.

2. The system of claim 1, wherein the microprocessor is configured to recalculate a new V based on fluorescence values of the previous n embryonic fish or juvenile aquatic organisms, or a portion thereof.

3. The system of claim 1, wherein c is from 2 to 10.

4. The system of claim 1, wherein n is from 10 to 10,000.

5. The system of claim 1, wherein the embryonic fish or juvenile aquatic organisms are aquaculturally farmed organisms.

6. The system of claim 1, wherein the embryonic fish are salmon, trout, zebrafish, wea bass, tilapia, catfish, sole, or cobia.

7. The system of claim 1, wherein the juvenile aquatic organisms are shrimp, lobster, prawn, crab or tiger prawn.

8. A computer implemented method for preparing groups of embryonic fish or juvenile aquatic organisms from a pool based on predicted growth potential or feed efficiency, said method comprising:
a. introducing to the pool of embryonic fish or juvenile aquatic organisms a colorimetric/fluorescent redox indicator, wherein fluorescence of the colorimetric/fluorescent redox indicator is for determining relative whole-body metabolic rate;
b. introducing the pool of embryonic fish or juvenile aquatic organisms to an automated system, wherein the automated system sequentially detects fluorescence values of n embryonic fish or juvenile aquatic organisms (E₁-Eₙ) and calculates a threshold fluorescence value V corresponding to a minimum fluorescence value within a X % highest fluorescence values detected, wherein n and X are pre-programmed by a user; and
c. sequentially detecting a fluorescence value of an additional embryonic fish or juvenile aquatic organism (Eₙ₊₁) and moving Eₙ₊₁ to a first container if the fluorescence value is greater than V or a container different from the first container if the fluorescence value is less than V.

9. The method of claim 8, wherein the method further comprises recalculating a new V based on fluorescence values of the previous n embryonic fish or juvenile aquatic organisms or a portion thereof.

10. The method of claim 9, wherein the method further comprises detecting a fluorescence value of a new embryonic fish or juvenile aquatic organism and moving the new embryonic fish or juvenile aquatic organism to a first container if the fluorescence value is greater than the new V or container different from the first container if the fluorescence value is less than the new V.

11. The method of claim 10, wherein the method repeats the steps:
a. recalculating a new V based on fluorescence values of the previous n embryonic fish or juvenile aquatic organisms or a portion thereof; and
b. detecting a fluorescence value of a new embryonic fish or juvenile aquatic organism and moving the new embryonic fish or juvenile aquatic organism to a first container if the fluorescence value is greater than the new V or a second container if the fluorescence value is less than the new V.

12. The method of claim 8, wherein the embryonic fish or juvenile aquatic organisms are aquaculturally farmed organisms.

13. The method of claim 8, wherein the embryonic fish are salmon, trout, zebrafish, wea bass, tilapia, catfish, sole, or cobia.

14. The method of claim 8, wherein the juvenile aquatic organisms are shrimp, lobster, prawn, or tiger prawn.

15. The method of claim 8, wherein n is from 10 to 100,000.

16. The method of claim 8, wherein the automated system comprises:
a. a detector for detecting a colorimetric/fluorescent redox indicator in an individual of the pool so as to measure a fluorescence value for the individual; and
b. a sorting component for moving an individual of the pool to one container out of c containers after detection of the redox indicator, wherein c is at least two; and
c. a microprocessor operatively connected to the detector and the sorting component, wherein the microprocessor receives input signals from the detector as fluorescence values of individuals of the pool and sends output signals to the sorting component to move the individuals to one of c containers, wherein the microprocessor is configured to:
i. receive and store fluorescence values from the detector as the detector sequentially detects light emitted from n embryonic fish or juvenile aquatic organisms (E₁ through Eₙ), wherein n is pre programmed by a user, wherein n is at least 10;
ii. organize a distribution of fluorescence values of all or a portion of the n detected embryonic fish or juvenile aquatic organisms;
iii. calculate V, wherein V is a minimum fluorescence value corresponding to the X % highest fluorescence values in the distribution, wherein X is pre-programmed by a user;
iv. receive and store a fluorescence value from the detector when the detector detects light emitted from a new embryonic fish or juvenile aquatic organism (Eₙ₊₁) and (i) send a first output signal to the sorting component to direct Eₙ₊₁ to a first container if the fluorescence value of Eₙ₊₁ is above V, or (ii) send a second output signal to the sorting mechanism to direct Eₙ₊₁ to a container different from the first container if the fluorescence value of Eₙ₊₁ is below V.

17. An automated computer implemented method for preparing groups of embryonic fish or juvenile aquatic organisms from a pool based on predicted growth potential or feed efficiency, said method comprising:
a. measuring whole-body metabolic rate of an individual in the pool of embryonic fish or juvenile aquatic organisms, wherein metabolic rate is measured by detection of a colorimetric/fluorescent redox indicator; and
b. moving the individual to a first container if the whole-body metabolic rate is above a precalculated threshold of fluorescence or moving the individual to a container different from the first container if the whole-body metabolic rate is below a precalculated threshold of fluorescence; and
c. repeating steps (a)-(b) for all of the individuals in the pool.

18. The method of claim 17, wherein the embryonic fish or juvenile aquatic organisms are aquaculturally farmed organisms.

19. The method of claim 17, wherein the embryonic fish are salmon, trout, zebrafish, wea bass, tilapia, catfish, sole, or cobia.

20. The method of claim 17, wherein the juvenile aquatic organisms are shrimp, lobster, prawn, or tiger prawn.
